(19) United States
(12) Patent Application Publication
(10) Pub. No.: US 2020/0000744 A1
(43) Pub. Date: Jan. 2, 2020

(54) PHARMACEUTICAL COMPOSITION

(71) Applicant: Avexxin AS, Trondheim (NO)

(72) Inventors: Charlotte Hessman, Malmö (SE); Birgitta Svennson, Malmö (SE); Anders Ljungqvist, Trondheim (NO)

(21) Appl. No.: 16/334,916

(22) PCT Filed: Sep. 21, 2017
(86) PCT No.: PCT/EP2017/073951

§ 371 Date: Mar. 20, 2019

(30) Foreign Application Priority Data
Sep. 21, 2016 (GB) 1616088.9

(51) Int. Cl. A61K 31/121; A61K 47/02; A61K 9/00; A61K 9/107; A61K 9/12; A61K 47/06; A61K 47/44

(52) U.S. Cl. CPC A61K 31/121; A61K 47/02; A61K 9/0014; A61K 47/44; A61K 9/12; A61K 47/06; A61K 9/107

(57) ABSTRACT

A pharmaceutical composition comprising polyunsaturated long-chain ketones and a silicone vehicle or a blend of silicone vehicles. The composition has utility in the treatment and prevention of inflammatory conditions including skin disorders.

This invention relates to a pharmaceutical composition comprising certain polyunsaturated long-chain ketones and a silicone vehicle, in particular a blend of silicone vehicles. The invention also relates to the use of said pharmaceutical formulation for the treatment or prevention of inflammatory conditions such as certain skin conditions, e.g. dermatitis and psoriasis.

### BACKGROUND

Certain polyunsaturated long chain ketones are described in various prior art references for the treatment of conditions including psoriasis, dermatitis, skin cancer, glomerulonephritis and rheumatoid arthritis (See EP-A-1469859, WO2010/139482, WO2012/028688, WO2014/082960 and WO2015/181135).

The polyunsaturated long chain ketones which are described in these references have amphiphilic character but are primarily hydrophobic and therefore insoluble in water. The lack of water solubility limits the bioavailability of the compounds and limits the ability of the skilled person to administer a useful dose of these compounds to a patient. In particular, the lack of water solubility limits the ability of the skilled person to administer the compounds topically to a patient.

A further problem with the polyunsaturated ketone compounds of the invention is that they are susceptible to degradation. Any formulation of these compounds should also ensure that the compounds remain stable for a prolonged period.

The present inventors sought to increase the permeation of the active polyunsaturated ketones into the skin whilst ensuring storage stability. It was surprisingly found that higher levels of permeation were observed when the polyunsaturated ketones were administered in a silicone-based vehicle. This enables a larger amount of active ingredient administered cutaneously to be absorbed into the skin. Moreover, the resulting compositions exhibit excellent storage stability (including chemical and physical stability). Without wishing to be bound to theory, the present inventors have found that the pharmaceutical compositions described herein typically form an oil-in-oil emulsion with the silicone-based vehicle which enhances use according to the invention.

### SUMMARY OF INVENTION

Thus, viewed from one aspect the invention provides a pharmaceutical composition comprising

(i) at least one compound of formula (I)

R-L-CO—CF₃ (I)

wherein R is an unsubstituted linear C₁₀₋₂₄ unsaturated hydrocarbon group, said hydrocarbon group comprising at least 4 non-conjugated double bonds;

L is a linking group forming a bridge of 2 to 5 atoms between the R group and the carbonyl CO wherein L comprises at least one of S, SO, SO₂ in the backbone of the linking group;

or a pharmaceutically acceptable salt, or a hydrate or solvate thereof; and at least one, preferably at least two, of

(ii) a cyclic silicone;
(iii) an elastomeric silicone; and
(iv) a liquid linear silicone.

In an exemplary embodiment, the silicone components (ii)-(iv) combined are present in an amount of at least 30 wt %, preferably at least 40 wt %, more preferably at least 50 wt %, e.g. at least 60 wt %, based on the total weight of the composition.

In particular, the invention relates to a pharmaceutical composition as herein before defined in which the compound of formula (I) is:

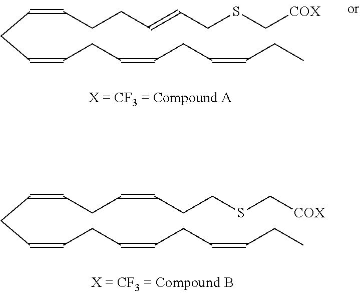

or a pharmaceutically acceptable salt, or a hydrate or solvate thereof.

Silicone vehicles having a range of viscosities (low to high) can be used according to the invention. In particular, the silicone vehicle preferably comprises two or more, especially all of the liquid silicone, cyclic silicone and elastomeric silicone. In a further embodiment, the composition of the invention can comprise other silicon based vehicles such as fumed silica compositions such as Aerosil® 200 and like compositions. Solid silicas, such as fumed silica, can be added to increase viscosity.

In particular, the silicone vehicle preferably comprises a cyclomethicone.

In particular, the silicone vehicle preferably comprises a cyclomethicone and an elastomeric silicone.

In particular, the silicone vehicle preferably comprises a liquid polydimethylsiloxane, a cyclomethicone and an elastomeric silicone.

Viewed from another aspect the invention provides a method of treating or preventing an inflammatory condition comprising administering to an animal, preferably a mammal, in need thereof, e.g. human, an effective amount of a composition as hereinbefore defined.

Viewed from another aspect the invention provides use of a composition as hereinbefore described in the manufacture of a medicament for use in the treatment or prevention of an inflammatory condition in an animal.

Viewed from another aspect the invention provides a composition as hereinbefore described for use in the treatment or prevention of an inflammatory condition in an animal.

Said condition is preferably a skin disorder such as psoriasis or dermatitis, such as atopic dermatitis.

The animal subject may be a mammal such as rodent (mouse, rat, rabbit), monkey (or other non-human primate), pig or other laboratory animal used as a model to study skin disorders or a human.

Viewed from another aspect the invention provides an article of manufacture comprising a container carrying the composition as hereinbefore defined.

The pharmaceutical compositions of the invention may be administered in a variety of different forms such as an emulsion, foam, ointment, gel, cream, as well as a spray, e.g. mist or aerosol. Topical administration is preferred.

### Definitions

The term “compound of the invention” relates to an active agent of formula (I), or a salt or solvate thereof, in particular a compound A or B as herein defined.

### DETAILED DESCRIPTION

This invention concerns a pharmaceutical composition comprising at least one compound of formula (I) and at least one silicone vehicle, preferably two, three, four or more of such silicone vehicles. In a preferred embodiment, the silicone vehicle comprises decamethylcyclopentasiloxane (cyclomethicone 5, D5), a liquid dimethicone and an elastomeric silicone. We have surprisingly found that the use of silicone vehicles results in an increased permeation into skin of the active ingredient of formula (I) when compared with formulations based on a paraffin vehicle. Our results demonstrate that the silicone based formulations generate better penetration of the active ingredient into porcine skin, resulting in a tissue concentration of active ingredient which is potentially 2 to 4 times better than paraffin-based formulations.

It is particularly preferred if the composition of the invention is in the form of an oil-in-oil emulsion. Without wishing to be bound to theory, it is believed that the oil-in-oil emulsion formed according to the invention is more physically stable if viscosity is increased, e.g. by including a suitable elastomeric silicone in the pharmaceutical composition. Moreover, such an emulsion also shows good penetration properties.

The silicone vehicles of use in the invention may not therefore dissolve the compound of formula (I). Rather the compound of formula (I) may be dispersed within the silicone vehicle.

### Pharmaceutical Composition of the Invention

The invention relies on the combination of at least one compound of formula (I) or a pharmaceutically acceptable salt, or a hydrate or solvate thereof and at least one, such as at least two silicone vehicles.

In a preferred embodiment, the composition comprises the compound of formula (I), a cyclomethicone and a dimethicone.

### Compounds of the Invention

The composition comprises at least one compound of formula (I):

R-L-CO—CF₃ (I)

Preferably, only one compound of formula (I) is present in the composition of the invention.

The group R preferably comprises 5 to 9 double bonds, preferably 5 to 8 double bonds, e.g. 5 to 7 double bonds such as 5 or 6 double bonds. These bonds should be non-conjugated. It is also preferred if the double bonds do not conjugate with the carbonyl functionality.

The double bonds present in the group R may be in the cis or trans configuration however, it is preferred if the majority of the double bonds present (i.e. at least 50%) are in the cis configuration. In further advantageous embodiments all the double bonds in the group R are in the cis configuration or all double bonds are in the cis configuration except the double bond nearest the carbonyl group which may be in the trans configuration.

The group R may have between 10 and 24 carbon atoms, preferably 17 to 19 carbon atoms.

The R group is unsubstituted. The R group is linear. It preferably derives from a natural source such as a long chain fatty acid or ester.

The linking group L provides a bridging group of 2 to 5 backbone atoms, preferably 2 to 4 backbone atoms between the R group and the carbonyl. The atoms in the backbone of the linker may be carbon and heteroatoms but will include at least one of S, SO, or SO₂. The linking group is preferably unsubstituted. It is preferably linear.

Preferred components of the linking group are —CH₂—, —S—, —SO—, and —SO₂— which can be combined with each other in any (chemically meaningful) order to form the linking group. Thus, by using two methylene groups and an —S— group the linker —SCH₂CH₂— is formed.

The linking group L contains at least one heteroatom in the backbone. It is also preferred if the first backbone atom of the linking group attached to the R group is a heteroatom or group of heteroatoms selected from —S—, —SO—, and —SO₂.

It is highly preferred if the linking group L contains at least one —CH₂— link in the backbone. Ideally the atoms of the linking group adjacent the carbonyl are —CH₂—.

It is preferred if a heteroatom —S—, —SO— or —SO₂ is positioned α, β, γ, or δ to the carbonyl, preferably β or γ to the carbonyl.

Highly preferred linking groups therefore are —SCH₂—, —SOCH₂—, or —SO₂CH₂—

Preferred compounds of formula (I) are those of formula (I′)

R—Y1-CH₂—CO—CF₃ (I′)

wherein R is as hereinbefore defined; and

Y1 is selected from S, SO or SO₂.

Highly preferred compounds for use in the invention are depicted below.

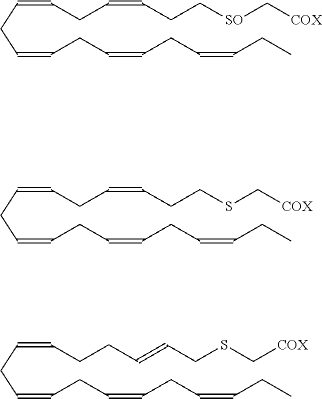

where X is CF₃.

The following compounds are highly preferred for use in the invention:

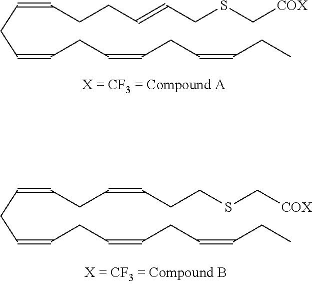

Where possible, the compounds can be present in the composition as a salt or solvate. Preferably however, no such form is used.

Compounds of formula (I) may be manufactured using known chemical synthetic routes found in J. Chem. Soc., Perkin Trans 1, 2000, 2271-2276 or J. Immunol., 1998, 161, 3421.

The polyunsaturated long chain ketones of the invention are preferably present in the formulation in an amount of from 0.1 wt % to 5.0 wt %, preferably 1.0 wt % to 4.0 wt %, e.g. about 3.0 wt %, based on the total weight of the formulation.

### Silicone Vehicle

The compositions of the invention preferably comprise a blend of silicone vehicles. By vehicle is meant a carrier or medium used as a diluent in which the medicinally active agent is formulated and/or administered.

The silicone vehicle may comprise cyclic silicones, liquid linear silicones and/or elastomeric silicones. By silicone is meant an oligo- or polysiloxane. In a preferred embodiment of the invention, the silicone vehicle comprises a cyclic silicone component and an elastomeric silicone component. In a further preferred embodiment of the invention, the silicone vehicle comprises a cyclic silicone component, a liquid linear silicone component, and an elastomeric silicone component. In a more preferred embodiment, the silicone vehicle comprises a cyclomethicone as the cyclic silicone component and a liquid polydimethylsiloxane as the liquid linear silicone component. The elastomeric silicone component may be a high molecular weight elastomeric silicone as found in commercially available silicone elastomer formulations, such as Dow Corning's Elastomer 10.

Cyclic Silicone

The cyclic silicone is preferably a cyclomethicone. Cyclomethicones are cyclic siloxanes of formula:

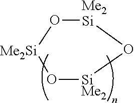

The cyclomethicones of the present invention preferably have n=1-5, even more preferably n=2-4, e.g. n=3. The cyclic silicones may thus be chosen from the group consisting of hexamethylcyclotrisiloxane (n=1), octamethylcyclotetrasiloxane (n=2), decamethylcyclopentasiloxane (n=3), dodecamethylcyclohexasiloxane (n=4) and tetradecamethylcycloheptasiloxane (n=5). In a preferred embodiment the pharmaceutical composition of the invention comprises decamethylcyclopentasiloxane (otherwise known as cyclomethicone 5, or D5). The cyclomethicones may be present in the composition either by addition of cyclomethicones in their pure form or by addition of commercial mixtures which contain cyclomethicones as one of their components. For example, Elastomer 10 by Dow Corning may be added which contains 87-88% cyclomethicone 5. The total amount of cyclomethicone present should be calculated from the amount of “pure” cyclomethicone added as well as from the amounts of cyclomethicone derived from other ingredients which contain cyclomethicone.

The cyclic silicone component is preferably present in an amount of at least 50 wt % based on the total weight of the composition. Preferably, the cyclic silicone is present in an amount of 60 to 99 wt %, preferably 70 to 95 wt %, e.g. 85 to 90 wt %, based on the total weight of the composition.

Liquid Linear Silicone

By liquid linear silicones is meant linear polysiloxanes which are liquid at 25° C. and ambient pressure. The term linear means that the siloxane is free of cyclic groups and side chain siloxane branches. In a preferred embodiment, the pharmaceutical composition of the invention comprises a liquid polydimethylsiloxane as said liquid linear silicone. Polydimethylsiloxanes are also known as dimethicones, and these two terms are hereafter used interchangeably. The commercially available polydimethylsiloxanes are often sold based on their viscosities, which are dependent on chain length. A low viscosity polydimethylsiloxane is needed to ensure that the dimethicone is a liquid.

The liquid linear siloxane of the invention may be a polydimethylsiloxane with an overall viscosity of 1 to 50 cSt, preferably 5 to 40 cSt, even more preferably 10 to 30 cSt. In a particularly preferred embodiment, the polydimethylsiloxane component has a viscosity of around 20 cSt. Commercially available Dimethicone 20 is thus suitable and is used in the exemplified embodiments of this invention.

The linear silicone liquid component is preferably present in an amount of 0.1 to 5.0 wt %, preferably 0.3 to 3.0 wt %, more preferably 0.5 to 1.5 wt % based on the total weight of the composition, e.g. about 1.0 wt % based on the total weight of the composition.

The use of dimethicone in the composition of the invention is valuable as cyclomethicone tends to evaporate in situ. The use of dimethicone ensures that a liquid remains on the skin together with any elastomer component. This enhances the dispersion of the active agent.

As dimethicone is not soluble in the cyclomethicone it is preferred to use the small percentages mentioned above to maximise its dispersion. The use of a low molecular weight dimethicone (i.e. low viscosity dimethicone) also enhances its dispersion in the cyclic silicone vehicle.

Elastomeric Silicone

The composition of the invention also preferably contains a silicone elastomer. These elastomers may be known as silicone rubbers. The elastomeric silicone may be a silicone elastomer which is for use as a vehicle, emollient and/or excipient in creams, ointments or any other topical pharmaceutical composition, such as that used in Elastomer 10. Often, the elastomeric silicone is also a PDMS but one having a much higher molecular weight and hence much higher viscosity than the liquid linear silicone. The viscosity of the combination of the elastomeric silicone with the cyclic silicone used in the present invention may be at least 10,000 cSt (cSt=centistokes), such as at least 100,000 cSt, more preferably at least 200,000 cSt, such as 350,000 Centistokes to 490,000 Centistokes. The elastomers themselves therefore have a viscosity of at least 500,000 Centistokes. The elastomers have very high viscosity and are often supplied in a silicone fluid. The elastomer may, for example, have a weight average molecular weight (Mw) of 200,000 or more, such as 250,000 to 900,000.

It is therefore possible for the composition of the invention to comprise a combination of polydimethylsiloxanes to achieve the desired composition. A combination of a liquid low viscosity and an elastomeric high viscosity polydimethylsiloxane may be used.

The silicone elastomer is preferably present in an amount of 1 to 20 wt %, preferably 3 to 15 wt % based on the total weight of the composition.

The silicone vehicle (i.e. the combination of all silicone components) is preferably present in an amount of at least 70 wt %, preferably at least 80 wt %, more preferably at least 90 wt %, e.g. at least 95 wt %, such as at least 99 wt % based on the total weight of the composition.

In a preferred embodiment, the invention provides a pharmaceutical composition comprising a compound of formula:

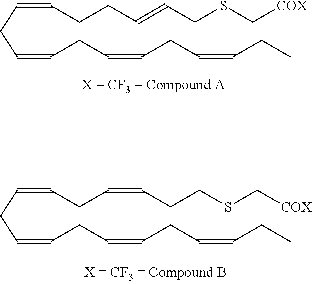

or a salt thereof; and

a silicone vehicle comprising a liquid polydimethylsiloxane, a cyclomethicone and an elastomeric silicone.

Ideally the composition should comprise about 70% (w/w) Elastomer 10. Ideally the composition should comprise about 1% dimethicone 20.

In a most preferred embodiment, the composition comprises a cyclic silicone in an amount of at least 50 wt % based on the total weight of the composition, preferably in an amount of 60 to 99 wt %, preferably 70 to 95 wt %, e.g. 85 to 90 wt %, based on the total weight of the composition;

an elastomeric silicone, preferably wherein said elastomeric silicone is present in an amount of 1 to 20 wt %, preferably 5 to 15 wt %, based on the total weight of the composition; and

a liquid linear silicone such as polydimethylsiloxane in an amount of 0.1 to 5 wt %, preferably 0.3 to 3 wt %, more preferably 0.5 to 1.5 wt % based on the total weight of the composition, e.g. about 1 wt % based on the total weight of the composition.

### Other Components

The compositions may also comprise an antioxidant.

The compositions may also comprise a silica, such as fumed silica. Such a component might form 1 wt % to 5.0 wt %, preferably 1.0 wt % to 4.0 wt %, e.g. about 3.0 wt %, based on the total weight of the formulation.

Formulations of the invention may be non aqueous. Traces of metals might encourage oxidative degradation of the compound of the invention so a chelating agent, e.g. EDTA or a salt thereof, may also be present. The compositions of the invention may also contain other active components, e.g. other drugs, although this is not preferred.

It is preferred if the compositions are free of paraffins. Ideally, the only excipients present are silicones (and optional antioxidants).

The composition is ideally in the form of an ointment, cream, salve or gel.

In a further preferred embodiment, the compositon of the invention can be administered as a spray, e.g. a mist of aerosol. It will be appreciated that a spray formulation requires a lower viscosity than, inter alia, a cream and hence the content of the higher viscosity elastomeric silicone may be reduced relative to other topical formulations.

Viewed from another aspect therefore, the invention provides a spray device comprising a pharmaceutical composition of the invention in a form suitable for spraying.

The viscosity of the sprayable composition may be 1 to 50 cSt, preferably 5 to 40 cSt, even more preferably 10 to 30 cSt.

Devices suitable for spraying a composition onto a skin surface are well known. Any atomising spray or aerosol type spray device can be used. The use of an aerosol or pump spray is preferred as this device also keeps the product in an air tight environment. Any spray device that can maintain an airtight environment is ideal.

The composition of the invention as a whole may have a viscosity of 1.0 to 300,000 cSt depending on the intended mechanism of application. For example, a spary may have a viscosity of 1 to 1000 cSt, a lotion a viscosity of 1000 to 100,000 cSt and a gel 10,000 to 300,000 cSt.

### Stability

The compounds of the invention can decompose into a variety of by-products. The generation of by-products can be reduced by formulation of the compounds as described herein.

As is illustrated by the examples, compositions of the invention have excellent long term storage stability. By “stable” it is meant that the purity area %, as measured by HPLC, is reduced by no more than 10% after 6 months of storage at 5° C., and preferably no more than 8.0% reduction after 6 months of storage at 5° C., preferably no more than 5.0% reduction after 6 months of storage at 5° C.

Preferably the peak area is reduced by no more than 20% after 6 months of storage at 25° C. Most preferably the peak area is reduced by no more than 10% after 6 months of storage at 25° C.

### Article

The compositions of the invention are suitable for administration to a patient. In order to administer the composition, it may be provided in a container that is essentially impermeable to that composition as well as air (particularly oxygen therein), as e.g. a collapsible, sealed aluminium tube, holding the composition. The container may form part of a kit along with instructions for administration of the composition. Where the administration route is topical, the container may be a squeezable tube or tub. Airless or essentially airless systems will be useful for some invention applications, for example, a pumpable bottle with airless pump. In one embodiment, a pump can be placed on a laminated aluminium tube and used accordingly.

As noted above, spray devices are also of interest.

Suitable containers may have a volume up to 100 ml, such as 5 to 100 ml.

### Treatment

The compositions of the invention are proposed for use in the treatment or prevention of inflammatory disorders including psoriasis, glomerulonephritis, lupus nephritis, diabetic nephropathy, rheumatoid arthritis or dermatitis. In particular, the condition to be treated is one that can be treated topically.

By treating or treatment is meant at least one of:

(i), inhibiting the disease i.e. arresting, reducing or delaying the development of the disease or a relapse thereof or at least one clinical or subclinical symptom thereof, or
(ii), relieving or attenuating one or more of the clinical or subclinical symptoms of the disease.

By prevention is meant (i) preventing or delaying the appearance of clinical symptoms of the disease developing in a mammal.

The benefit to a subject to be treated is either statistically significant or at least perceptible to the patient or to the physician. In general a skilled man can appreciate when “treatment” occurs. It is particularly preferred if the composition of the invention are used therapeutically, i.e. to treat a condition which has manifested rather than prophylactically. It may be that the composition of the invention is more effective when used therapeutically than prophylactically.

The composition of the invention can be used on any animal subject, in particular a mammal and more particularly to a human or an animal serving as a model for a disease (e.g., mouse, monkey, etc.).

In order to treat a disease an effective amount of the active composition needs to be administered to a patient. A “therapeutically effective amount” means the amount of a composition that, when administered to an animal for treating a state, disorder or condition, is sufficient to effect such treatment. The “therapeutically effective amount” will vary depending on the composition, the disease and its severity and the age, weight, physical condition and responsiveness of the subject to be treated and will be ultimately at the discretion of the attendant doctor.

It will be appreciated that pharmaceutical composition for use in accordance with the present invention is ideally in a form for topical administration, e.g. as an ointment, gel, salve or cream.

Therapeutic doses will generally be between about 0.5 and 2000 mg/day, for example, between about 1-10 mg/day up to 1500 mg/day. Other ranges may be used, including, for example, 6-, 7-, 8-, 9-, 10-500 mg/day, 50-300 mg/day, 100-200 mg/day.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art.

### DESCRIPTION OF FIGURES

FIG. 1 shows the tissue concentration of Compound A in the porcine ear dermis after 24 h using PBS as receptor solution.

FIG. 2 shows the tissue concentration of Compound A in the porcine ear stratum corneum and epidermis after 24 h using PBS as receptor solution.

FIG. 3 shows the mean percent penetrated Compound A (μg/cm²×h) at 6 hours penetration (95% confidence interval).

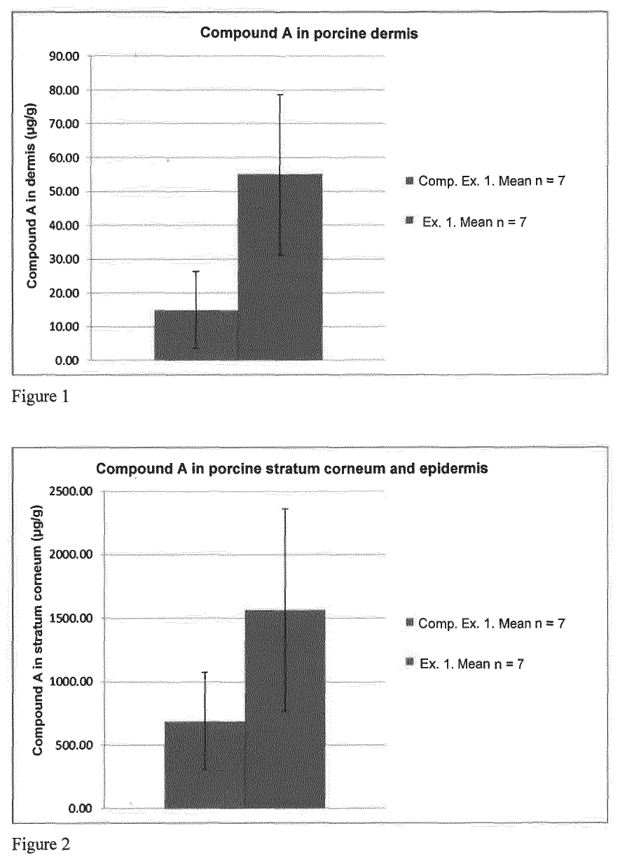

### EXAMPLES

The following compounds were used in the Experiments:

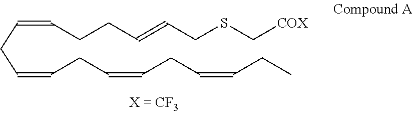

### Example 1—In Vitro Penetration of 3% Compound a Silicone Formulation Through Porcine Skin

### Experimental

The penetration of Compound A from a silicone-based formulation containing 3% Compound A through fresh porcine skin was compared with a petrolatum/paraffin-based formulation under analogous conditions. The accumulated amount of Compound A was analysed in stratum corneum/epidermis and dermis after 24 hours.

### Compositions

TABLE 1
Inventive and comparative formulations

| | Comparative | |
|---|---|---|
| | Example 1 | Example 1 |
| Raw material | % (w/w) | % (w/w) |
| Compound A | 3.2 | 3.2 |
| Petrolatum (paraffin, with soft) | 77.4 | — |
| Liquid Paraffin | 19.4 | — |
| Elastomer 10 (with about 87-88% | — | 72.6 |
| Cyclomethicone 5-NF) | | |
| Cyclomethicone 5 | — | 23.2 |
| Dimethicone 20 | — | 1.0 |
| Sum (%) | 100 | 100 |

### Methods

TABLE 2
Experimental parameters for Bronaugh cell experiment

| Parameter | Value/Interval |
|---|---|
| Receptor solution | PBS pH 7.4 |
| Receptor flow | About 1.6 ml/min |
| Light conditions | Ambient, no precaution was taken |
| T_{receptor phase} | 34.5° C. giving the cell temperature 32.0° C. |
| Membrane | Full thickness porcine ear skin |
| Application interval | One application |
| Applied amount | Applied amount 50 mg formulation/cell. |
| Application | The formulation was applied using a spatula. |
| procedures | |
| Sampling interval | 24 hours. |
| Duration of study | 24 h |

Washout phase

| | |
|---|---|
| Wash procedure | Formulation was removed with receptor |
| | solution using tops. |

The fresh porcine ear skin used in the study was pre-dermatomed to about 0.5-1 mm thick.

### Results:

TABLE 3

| | | | Applied | Applied | Membrane |
|---|---|---|---|---|---|
| | | | dose | active | thickness |
| Cell | Membrane type | Formulation | (mg) | (μg) | (mm) |
| 1 | Fresh pig ear skin | Comp Ex 1 | 54.2 | 499.5 | 0.840 |
| 2 | Fresh pig ear skin | Comp Ex 1 | 57.4 | 443.7 | 0.710 |
| 3 | Fresh pig ear skin | Comp Ex 1 | 55.0 | 455.4 | 0.787 |
| 4 | Fresh pig ear skin | Comp Ex 1 | 55.9 | 475.2 | 0.713 |
| 5 | Fresh pig ear skin | Comp Ex 1 | 56.2 | 463.5 | 0.757 |
| 6 | Fresh pig ear skin | Comp Ex 1 | 51.8 | 1437.0 | 0.796 |
| 7 | Fresh pig ear skin | Comp Ex 1 | 53.4 | 1587.0 | 0.711 |
| 8 | Fresh pig ear skin | Ex 1 | 54.0 | 1542.0 | 0.776 |
| 9 | Fresh pig ear skin | Ex 1 | 59.3 | 1695.0 | 0.925 |
| 10 | Fresh pig ear skin | Ex 1 | 55.7 | 1635.0 | 0.873 |
| 11 | Fresh pig ear skin | Ex 1 | 54.3 | 2395.0 | 0.879 |
| 12 | Fresh pig ear skin | Ex 1 | 56.0 | 2475.0 | 0.927 |
| 13 | Fresh pig ear skin | Ex 1 | 51.6 | 2670.0 | 0.877 |
| 14 | Fresh pig ear skin | Ex 1 | 59.9 | 2530.0 | 0.884 |

TABLE 4
Tissue analysis of Compound A after 24 h

Tissue concentrations
24 hours penetration

| Fresh porcine | Amount A statum | Amount A |
|---|---|---|
| skin, cell no. | corneum (μg/g) | dermis (μg/g) |
| 1 | 820.88 | 8.73 |
| 2 | 380.82 | 7.27 |
| 3 | 1391.31 | 33.36 |
| 4 | 407.97 | 5.04 |
| 5 | 944.31 | 29.20 |
| 6 | 458.69 | 8.90 |
| 7 | 409.15 | 12.22 |
| Mean n = 7 | 687.59 | 14.96 |
| 8 | 919.43 | 37.36 |
| 9 | 1526.01 | 86.25 |
| 10 | 2833.50 | 75.15 |
| 11 | 2516.73 | 67.62 |
| 12 | 1247.06 | 49.47 |
| 13 | 836.53 | 16.65 |
| 14 | 1068.30 | 52.44 |
| Mean n = 7 | 1563.94 | 54.99 |

TABLE 5
Results from tissue analysis of Compound A after 24 h.

| | | Amount | | | | |
|---|---|---|---|---|---|---|
| | Dos | stratum | Skin wash | Skin | Amount | Total |
| Cell | applied | corneum | fraction | edge | dermis | absorbed |
| no. | (μg) | (μg) | μg) | (μg) | (μg) | (μg) |
| 1 | 1626 | 4.137 | 1078.606 | 1.827 | 0.418 | 2.245 |
| 2 | 1722 | 1.919 | 665.309 | 0.412 | 0.289 | 0.701 |
| 3 | 1650 | 7.012 | 1046.151 | 0.538 | 1.486 | 2.024 |
| 4 | 1677 | 2.056 | 704.435 | 2.350 | 0.201 | 2.551 |
| 5 | 1686 | 4.759 | 505.158 | 6.689 | 1.246 | 7.934 |
| 6 | 1554 | 2.312 | 725.044 | 2.388 | 0.402 | 2.790 |
| 7 | 1602 | 2.062 | 760.019 | 0.440 | 0.486 | 0.926 |
| Mean | 1645 | 3.977 | 799.932 | 2.092 | 0.647 | 3.091 |
| 8 | 1620 | 4.634 | 1337.228 | 3.271 | 1.685 | 4.956 |
| 9 | 1779 | 7.691 | 1302.327 | 4.223 | 3.429 | 7.652 |
| 10 | 1671 | 14.281 | 1471.339 | 6.172 | 3.295 | 9.467 |
| 11 | 1629 | 12.684 | 1575.068 | 11.804 | 3.600 | 15.404 |
| 12 | 1680 | 6.285 | 1321.360 | 0.313 | 2.471 | 2.784 |
| 13 | 1548 | 4.216 | 1313.681 | 1.393 | 0.838 | 2.232 |
| 14 | 1797 | 5.384 | 1521.596 | 1.981 | 2.798 | 4.780 |
| Mean | 1674 | 7.882 | 1406.085 | 4.165 | 2.588 | 6.75 |

Conclusion: An in vitro permeability study using fresh porcine skin to compare a modified silicone based formulation containing 3% (w/w) of Compound A with the reference petrolatum based 3% Compound A formulation was performed. The silicone based formulation generated higher tissue concentration of Compound A than the originator petrolatum based formulation.

### Example 2

Testing in Silicon Sheeting: An in vitro study was carried out using a silicone membrane. The following test formulations were used:

TABLE 6

| | Comp. Ex 1 | Comp Ex 2 | Ex 1 |
|---|---|---|---|
| | % (w/w) | % (w/w) | % (w/w) |
| Compound A | 3.2 | 3.2 | 3.2 |
| Petrolatum (paraffin, with | 77.4 | 72.6 | — |
| soft) | | | |
| Liquid Paraffin | 19.4 | — | — |
| Elastomer 10 (with about | — | 24.2 | 72.6 |
| 87-88% Cyclomethicone | | | |
| 5-NF) | | | |
| Cyclomethicone 5 | — | — | 23.2 |
| Dimethicone 20 | — | — | 1.0 |
| | 100 | 100 | 100 |

TABLE 7
Experimental parameters for Franz cell experiment:

| Parameter | Value/Interval |
|---|---|
| Receptor phase | 50% ethanol in water. |
| composition | |
| Receptor volume | About 7 ml |
| Cell area | 2.01 cm² |
| Light conditions | Ambient, no percussion was taken |
| T_{receptor phase} | 32° C. |
| Membrane | 30 mm, Silicon sheeting 0.005″ NRV M/M40D |
| Application | One application |
| interval | |
| Applied amount | Applied amount 400 mg formulation/cell. |
| Application | The formulation will be applied using syringe. |
| procedures | |
| Sampling interval | 6 hours. |
| Sampling volume | The sampling was made from the centre of |
| | the Franz cell. Sample volume 400 μl. |
| Duration of study | 6 h |

Washout phase

Briefly, the Franz Cell chamber is an in vitro skin permeation assay frequently used in formulation development. The Franz Cell apparatus consists of two primary chambers separated by a membrane. Animal and human skin can be used as the membrane. The test product is applied to the membrane via the top chamber. The bottom chamber contains fluid from which samples are taken at regular intervals for analysis. This testing determines the amount of active that has permeated the membrane at each time point. The chamber is maintained at a constant temperature of 37° C. As such, the Franz Cell analysis allow comparison whether a particular formulation delivers an active agent through the skin.

The membranes were washed with RO water prior to use.

TABLE 8
Experimental set up 1.

| | Cell 1 | Cell 2 | Cell 3 | Cell 4 | Cell 5 | Cell 6 | Cell 8 | Cell 9 | Cell 10 |
|---|---|---|---|---|---|---|---|---|---|
| Formulation | CE1 | | | CE2 | | | Ex 1 | | |
| Sample | 412.3 | 389.9 | 394.3 | 409.9 | 417.5 | 395.6 | 391.6 | 414.0 | 409.5 |
| weight (mg) | | | | | | | | | |

| | | | |
|---|---|---|---|
| Time (h) | 6 | 6 | 6 |

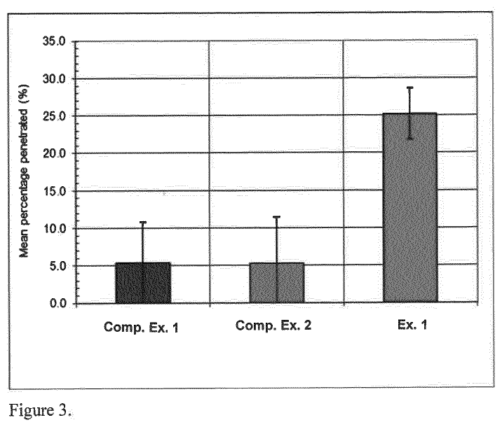

Results are presented in FIG. 3 and table 9.

TABLE 9
Cumulative penetration of Compound A solution
through silicon sheeting after 6 h.

| | Mean cumulative |
|---|---|
| | penetrated Compound |
| Sample | A of applied dose (%) |
| CE1 | 5.35 |
| CE2 | 5.29 |
| Ex 1 | 25.20 |

The example formulation showed significantly higher sheet penetration in this model.

### Example 3: Stability Data

The stability of the 3% silicone-based Compound A composition above was tested at 5 and 25° C. The product was filled in 5 g sealed aluminium tubes, lacquered on the inside with and epoxy/phenol lacquer. The levels of impurities formed during storage of a batch of the same composition as example 1 were monitored at T=0 months, T=1 month, T=3 months and T=6 months.

TABLE 10
Ex 1 stored at 5° C.

| | | T = 1 | T = 3 | T = 6 |
|---|---|---|---|---|
| Test | T = 0 | month | months | months |
| Appearance | Complies | Complies | Complies | Complies |
| Assay (w/w %) | 101% | 96% | 90 | 88 |
| Purity (area %) | 91.6% | 93.4% | 89.7 | 92.3 |
| Specified Impurities | | | | |
| Single: | | | | |
| Approx. RRT = 0.56 | 3.6% | 2.3% | 5.7% | 3.2% |
| Approx. RRT = 0.96 | 0.3% | 0.3% | 0.3% | 0.3% |
| Approx. RRT = 1.02 | Not | Not | 0.4% | 0.2% |
| | Detected | Detected | | |
| Approx. RRT = 1.03 | 1.0% | 0.6% | Not | Not |
| | | | Detected | Detected |
| Approx. RRT = 1.06 | 0.2% | 0.2% | 0.3% | 0.4% |
| Approx. RRT = 1.22 | 0.1% | 0.1% | 0.1% | 0.1% |
| Approx. RRT = 1.43 | 1.4% | 1.2% | 1.0% | 0.8% |
| Total: | 6.6% | 4.7% | 7.8% | 5.0% |
| Unspecified Imp.: | | | | |
| Single: | | | | |
| Approx. RRT = 0.72 | 0.2% | 0.2% | 0.3% | 0.4% |
| Approx. RRT = 0.90 | Not | 0.1% | 0.1% | 0.1% |
| | Detected | | | |
| Approx. RRT = 1.50 | 0.2% | <0.1% | Not | Not |
| | | | Detected | Detected |
| Approx. RRT = 2.32 | Not | Not | 0.3% | 0.2% |
| | Detected | Detected | | |
| Approx. RRT = 2.41 | 0.2% | Not | Not | Not |
| | | Detected | Detected | Detected |
| Approx. RRT = 2.62 | Not | Not | 0.7% | Not |
| | Detected | Detected | | Detected |
| Approx. RRT = 2.74 | Not | Not | 0.8% | Not |
| | Detected | Detected | | Detected |
| Approx. RRT = 3.10 | 0.5% | 0.6% | Not | Not |
| | | | Detected | Detected |
| Approx. RRT = 3.18 | 0.8% | 0.8% | Not | Not |
| | | | Detected | Detected |
| Approx. RRT = 3.36 | Not | 0.1% | Not | Not |
| | Detected | | Detected | Detected |
| Other | | | 0.3% | 2.2% |
| Total: | 1.9% | 1.8% | 2.5% | 2.9% |

Based on the data obtained in this exploratory study at refrigerator and accelerated temperature, storage for at least 6 months at 2-8° C. can be expected for the Example 1 formulation.

TABLE 11
Ex 1 stored at 25° C.

| | | T = 1 | T = 3 | T = 6 |
|---|---|---|---|---|
| Test | T = 0 | month | months | months |
| Appearance | Complies | Complies | Complies | Complies |
| Assay (w/w %) | 101% | 98% | 89% | 89% |
| Purity (area %) | 91.6% | 94.2% | 91.8% | 92.3% |
| Specified Impurities | | | | |
| Single: | | | | |
| Approx. RRT = 0.56 | 3.6% | 0.7% | 1.1% | 0.3% |
| Approx. RRT = 0.96 | 0.3% | 0.3% | 0.3% | 0.3% |
| Approx. RRT = 1.02 | Not | Not | 0.5% | 0.3% |
| | Detected | Detected | | |
| Approx. RRT = 1.03 | 1.0% | 0.6% | | |
| Approx. RRT = 1.06 | 0.2% | 1.2% | 1.4% | 1.5% |
| Approx. RRT = 1.22 | 0.1% | 0.1% | 0.2% | 0.1% |
| Approx. RRT = 1.43 | 1.4% | <0.1% | 0.2% | 0.1% |
| Total: | 6.6% | 2.9% | 3.7% | 2.7% |
| Unspecified Imp.: | | | | |
| Single: | | | | |
| Approx. RRT = 0.72 | 0.2% | 1.3% | 2.3% | 2.3% |
| Approx. RRT = 0.90 | 0.2% | <0.1% | 0.1% | 0.1% |
| Approx. RRT = 1.50 | | Not | | |
| | | Detected | | |
| Approx. RRT = 2.32 | | | 0.5% | |
| Approx. RRT = 2.41 | 0.2% | | | |
| Approx. RRT = 2.62 | | | 0.7% | |
| Approx. RRT = 2.74 | | | 0.8% | |
| Approx. RRT = 3.10 | 0.5% | 0.6% | | |
| Approx. RRT = 3.18 | 0.8% | 0.8% | | |
| Other | | | 0.3% | 2.7% |
| Total: | 1.9% | 2.7% | 4.6% | 5.1% |

### Example 4

A second test has been carried out using the following formulation:

TABLE 12

| | Example 2 |
|---|---|
| Raw material | % (w/w) |
| Compound A | 1.1 |
| Elastomer 10 (with about 87-88% | 73.9 |
| Cyclomethicone 5-NF) | |
| Cyclomethicone 5 | 24.0 |
| Dimethicone 20 | 1.0 |
| Sum (%) | 100 |

The stability of the Example 2 composition above was tested at 25° C. The product was filled in 5 g sealed aluminium tubes, lacquered on the inside with and epoxy/phenol lacquer. The levels of impurities formed during storage of a batch of the same composition as example 1 were monitored at T=0 months, T=1 month, T=2 months and T=3 months.

TABLE 13
stability data for Example 2 formulation.

| | | T = 1 | T = 2 | T = 3 |
|---|---|---|---|---|
| Test | T = 0 | month | months | months |
| Appearance | Complies | Complies | Complies | Complies |
| Purity (area %) | 92.9% | 90.7% | 89.5% | 89.5% |
| Specified Impurities | | | | |
| Single: | | | | |
| Approx. RRT = 0.56 | 1.4% | 2.0% | 2.1% | 1.9% |
| Approx. RRT = 0.96 | 0.4% | 0.4% | 0.3% | 0.3% |
| Approx. RRT = 1.02 | Not | Not | Not | Not |
| | Detected | Detected | Detected | Detected |
| Approx. RRT = 1.03 | 2.5% | 2.7% | 3.0% | 3.1% |
| Approx. RRT = 1.06 | 0.2% | 0.5% | 0.6% | 0.6% |
| Approx. RRT = 1.22 | 0.2% | 0.2% | 0.2% | 0.2% |
| Approx. RRT = 1.43 | 0.2% | 0.1% | 0.1% | 0.1% |
| Total: | 4.9% | 5.9% | 6.3% | 5.8% |
| Unspecified Imp.: | | | | |
| Single: | | | | |
| Approx. RRT = 0.72 | 0.1% | 1.0% | 1.7% | 1.9% |

As can be seen in Table 13, the purity of the composition decreased only very slightly from 92.9% to 89.5% over the course of three months storage at 25° C. Based on the data obtained in this exploratory study, storage for at least 6 months at 2-8° C. can be expected for the Example 2 formulation.

### Example 5

Testing in Silicon Sheeting: An in vitro study is carried out using a silicone membrane. The following test formulations are used:

TABLE 14

| | Ex 3 | Ex 4 |
|---|---|---|
| | % (w/w) | % (w/w) |
| Compound A | 3.2 | 3.2 |
| Petrolatum (paraffin, with | 10 | 5 |
| soft) | | |
| Liquid Paraffin | — | — |
| Elastomer 10 (with about | 62 | 67 |
| 87-88% Cyclomethicone | | |
| 5-NF) | | |
| Cyclomethicone 5 | 23.8 | 23.8 |
| Dimethicone 20 | 1.0 | 1.0 |
| Sum (%) | 100 | 100 |

Cumulative penetration of Compound A can be measured in the Franz cell experiment (table 7) and can be determined according to standard procedures that determine the mean cumulative penetration of Compound A as a percentage of applied dose. The formulation giving the best cumulative penetration will be preferred for many applications of the invention.

### Example 6

A further stability test has been carried out using the formulations below:

TABLE 15

% (w/w)

| Ingredient | Ex 1 | Comparative Ex 3 |
|---|---|---|
| Compound A | 3.2 | 0 |
| Elastomer 10 | 72.6 | 75.0 |
| Cyclomethicone 5 | 23.2 | 24 |
| Dimethicone 5 | 1.00 | 1.00 |
| Sum (%) | 100.0 | 100.0 |

The stability of the 3% silicone-based Compound A composition above was tested at 5 and 25° C. The product was filled in 5 g sealed aluminium tubes, lacquered on the inside with and epoxy/phenol lacquer. The levels of impurities formed during storage of a batch of the same composition as example 1 were monitored (by HPLC) at T=0 months, T=1 month, T=3 months, T=6 months and T=12 months. The appearance of both compositions, and the content and purity of the Ex 1 composition are reported. The results are reported in Tables 16 to 18.

TABLE 16
Ex 1 stored at 5° C.

| | Time (months) | | | | | |
|---|---|---|---|---|---|---|
| | 0 | 1 | 3 | 6 | 9 | 12 |
| Appearance | Clear, | Slightly | Clear, | Slightly | Slightly | Slightly |
| | colourless | translucent, | colourless | translucent, | translucent, | translucent, |
| | | colourless | | colourless | colourless | colourless |
| Assay (content of | 103 | 102 | 97 | 100 | 99 | 102 |
| Compound A 100%) | | | | | | |
| Purity (area %) | 96 | 95 | 96 | 96 | 96 | 96 |
| Specified | | | | | | |
| Impurities(area) | | | | | | |
| RRT Approx. Sum | 2.0 | 2.2 | 2.0 | 1.3 | 1.1 | 1.3 |
| 0.54-0.56 | | | | | | |
| RRT Approx. 0.96 | ND | ND | ND | ND | ND | ND |
| RRT Approx. 1.02 | 0.4 | 0.4 | 0.3 | 0.2 | 0.2 | 0.3 |
| RRT Approx. 1.03 | ND | ND | ND | ND | ND | ND |
| RRT Approx 1.06 | 0.1 | 0.1 | 0.1 | 0.2 | 0.2 | 0.3 |
| RRT Approx 1.22 | 0.1 | 0.1 | <0.1 | <0.05 | 0.1 | 0.1 |
| RRT Approx 1.43 | 0.4 | 0.4 | 0.3 | 0.2 | 0.1 | ND |
| Total specified | 3.0 | 3.2 | 2.7 | 1.9 | 1.7 | 1.9 |
| impurities | | | | | | |
| Total | 1.1 | 1.5 | 1.5 | 1.5 | 1.9 | 2.2 |
| unspecified | | | | | | |
| impurities | | | | | | |

TABLE 17
Ex 1 stored at 25° C.

| | Time (months) | | | | | |
|---|---|---|---|---|---|---|
| | 0 | 1 | 3 | 6 | 9 | 12 |
| Appearance | Clear, | Slightly | Clear, | Slightly | Slightly | Slightly |
| | colourless | translucent, | colourless | translucent, | translucent, | translucent, |
| | | colourless | | colourless | colourless | colourless |
| Assay (content of | 103 | 101 | 98 | 101 | 100 | 102 |
| Component A 100%) | | | | | | |
| Purity (area %) | 96 | 96 | 96 | 97 | 96 | 97 |
| Specified | | | | | | |
| Impurities(area) | | | | | | |
| RRT Approx. Sum | 2.0 | 1.0 | 0.4 | 0.4 | 0.2 | 0.2 |
| 0.54-0.56 | | | | | | |
| RRT Approx. 0.96 | ND | ND | ND | ND | ND | ND |
| RRT Approx. 1.02 | 0.4 | 0.4 | 0.3 | 0.2 | 0.2 | 0.3 |
| RRT Approx. 1.03 | ND | ND | ND | ND | ND | ND |
| RRT Approx 1.06 | 0.1 | 0.4 | 0.4 | 0.3 | 0.4 | 0.3 |
| RRT Approx 1.22 | 0.1 | 0.1 | 0.1 | 0.1 | 0.1 | 0.1 |
| RRT Approx 1.43 | 0.4 | 0.1 | ND | ND | ND | ND |
| Total specified | 3.0 | 1.9 | 1.1 | 1.0 | 0.8 | 0.8 |
| impurities | | | | | | |
| Total | 1.1 | 2.1 | 2.4 | 2.2 | 3.2 | 2.5 |
| unspecified | | | | | | |
| impurities | | | | | | |

TABLE 18
Comp Ex 3

| | Time (months) | | | | | |
|---|---|---|---|---|---|---|
| | 0 | 1 | 3 | 6 | 9 | 12 |
| Appearance | Slightly | Slightly | Clear, | Slightly | Slightly | Slightly |
| (5° C.) | translucent | translucent | colourless | translucent, | translucent, | translucent, |
| | colourless | colourless | | colourless | colourless | colourless |
| | ointment | ointment | | | | |
| Appearance | Slightly | Slightly | Clear, | Slightly | Slightly | Slightly |
| (25° C.) | translucent | translucent | colourless | translucent, | translucent, | translucent, |
| | colourless | colourless | | colourless | colourless | colourless |
| | ointment | ointment | | | | |

1. A pharmaceutical composition comprising:
(i) at least one compound of formula (I)
R-L-CO—CF₃ (I)
wherein R is an unsubstituted linear C₁₀₋₂₄ unsaturated hydrocarbon group, said hydrocarbon group comprising at least 4 non-conjugated double bonds;
L is a linking group forming a bridge of 2 to 5 atoms between the R group and the carbonyl CO wherein L comprises at least one of S, SO, SO₂ in the backbone of the linking group;
or a pharmaceutically acceptable salt, or a hydrate or solvate thereof; and at least one, preferably at least two of
(ii) a cyclic silicone;
(iii) an elastomeric silicone; and
(iv) a liquid linear silicone.

2. The composition of claim 1 wherein the silicone components (ii)-(iv) combined are present in an amount of at least 30 wt % based on the total weight of the composition.

3. The composition of claim 1, comprising a cyclic silicone in an amount of at least 50 wt % based on the total weight of the composition, preferably in an amount of 60 to 99 wt %, preferably 70 to 95 wt %, e.g. 85 to 90 wt %, based on the total weight of the composition.

4. The composition of claim 1, wherein the cyclic silicone is selected from the group consisting of hexamethylcyclotrisiloxane, octamethylcyclotetrasiloxane, decamethylcyclopentasiloxane, dodecamethylcyclohexasiloxane and tetradecamethylcycloheptasiloxane, preferably decamethylcyclopentasiloxane (cyclomethicone 5).

5. The composition of claim 1 comprising an elastomeric silicone, preferably wherein said elastomeric silicone is present in an amount of 1 to 20 wt %, preferably 5 to 15 wt %, based on the total weight of the composition.

6. The composition of claim 1, wherein the elastomeric silicone is a silicone elastomer formulation which is for use as a vehicle, emollient and/or excipient in creams, ointments or any other topical pharmaceutical composition, such as Elastomer 10.

7. The composition of claim 1, wherein the viscosity of the combination of the elastomeric silicone with the cyclic silicone is at least 10,000 cSt (cSt=centistokes), such as at least 100,000 cSt, more preferably at least 200,000 cSt, such as 350,000 Centistokes to 490,000 Centistokes.

8. The composition of claim 1 comprising polydimethylsiloxane as said liquid linear silicone, e.g. in an amount of 0.1 to 5 wt %, preferably 0.3 to 3 wt %, more preferably 0.5 to 1.5 wt % based on the total weight of the composition, e.g. about 1 wt % based on the total weight of the composition.

9. The composition of claim 8, wherein the viscosity of the polydimethylsiloxane is of 1 to 50 cSt, preferably 5 to 40 cSt, even more preferably 10 to 30 cSt, such as 20 cSt, preferably dimethicone 20.

10. The composition of claim 1, wherein the silicone components (ii)-(iv) combined are present in an amount of at least 70 wt %, preferably at least 80 wt %, more preferably at least 90 wt %, e.g. at least 95 wt %, based on the total weight of the composition.

11. The composition of claim 1, wherein the compound of formula (I) is of formula (I′)
R—Y1-CH₂—CO—CF₃ (I′)
wherein R is as defined in claim 1; and
Y1 is selected from S, SO or SO₂, preferably

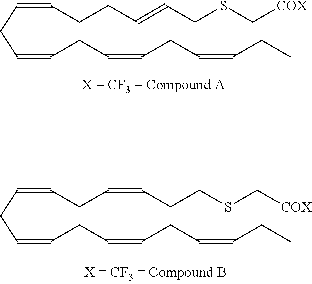

12. The composition of claim 1, wherein the compound of formula (I) is present in an amount of from 0.1 wt % to 5.0 wt %, preferably 1.0 wt % to 4.0 wt %, e.g. about 3.0 wt %, based on the total weight of the formulation.

13. The composition of claim 1, further comprising an antioxidant.

14. The composition of claim 1, wherein the purity area % of the compound of formula (I), as measured by HPLC, is reduced by no more than 10% after 6 months of storage at 5° C., and preferably no more than 8.0% reduction after 6 months of storage at 5° C., preferably no more than 5.0% reduction after 6 months of storage at 5° C. in an inert atmosphere.

15. The composition of claim 1 in a form suitable for topical administration.

16. The composition of claim 1 in the form of an oil-in-oil emulsion.

17. An article of manufacture comprising a container holding the pharmaceutical composition of claim 1 and optionally, directions for using the composition.

18. An article as claimed in claim 17 which is a spray device.

19. (canceled)

20. (canceled)

21. A method of treating or preventing an inflammatory condition comprising administering to an animal, preferably a mammal, in need thereof, e.g. human, an effective amount of the composition as defined in claim 1.
